(19) United States
(12) Patent Application Publication
(10) Pub. No.: US 2007/0161990 A1
(43) Pub. Date: Jul. 12, 2007

(54) Spinal implant hooks and systems

(75) Inventors: Angela L. Hillyard, Greenfield, MN (US); Paul F. Boschert, Minneapolis, MN (US); Jeffrey Cota, St. Hilaire, MN (US); Mike E. Lancial, St. Louis Park, MN (US); Duy Nguyen, Corona, CA (US)

Correspondence Address: WOOD, HERRON & EVANS (ZIMMER SPINE), 2700 CAREW TOWER, 441 VINE STREET, CINCINNATI, OH, 45202, US

(73) Assignee: Zimmer Spine, Inc., Minneapolis, MN (US)

(21) Appl. No.: 11/314,217

(22) Filed: Dec. 21, 2005

(51) Int. Cl. A61F 2/30

(52) U.S. Cl. 606/61

(57) ABSTRACT

Bone hooks for use in a spinal fixation system are provided. A device of the invention includes a head portion that can engage a spinal rod of a spinal fixation system, and a hook portion that can be positioned on and/or around a portion of a vertebra. In one embodiment of the invention, the hook portion is adjustably movable with respect to the body portion. In another embodiment, the hook portion includes an auxiliary bone engaging element that can contact a vertebra positioned with a bone receiving region of the hook portion. The auxiliary bone engaging element according to the present invention is particularly useful for maintaining the desired position of the device both during and after the implantation of a spinal fixation system.

### TECHNICAL FIELD

The present invention relates generally to devices for use in spinal surgery and methods of using such devices. More particularly, the present invention relates to bone hooks for anchoring a vertebral body to a spinal fixation system as part of a surgical procedure.

### BACKGROUND

Spinal fixation systems are used in orthopedic surgery to correct a variety of back structure problems, such as those that occur as a result of trauma or improper development during growth. Generally, these systems correct such problems by providing a desired corrective spatial relationship between vertebral bodies. Such systems typically include one or more fixation rods that are coupled to adjacent vertebra by attaching the rods to various anchoring devices, such as hooks, bolts, wires, or screws. The fixation rods have a predetermined contour designed according to a particular implantation site, and once installed, the fixation system holds the vertebral bodies in a desired spatial relationship, either until desired healing or spinal fusion has taken place, or for some longer period of time.

Bone anchors such as screws and hooks are commonly utilized to facilitate segmental attachment of such connective structures to the posterior surfaces of the spinal laminae. Because each vertebra varies in shape and size, a variety of anchoring devices have been developed to facilitate attachment with a particular portion of the bone. Bone screws are typically implanted through the pedicles and into the vertebral bodies. Bone hooks are typically hooked around the laminae or other spinal processes. These components are intended to provide the necessary stability both in tension and compression and to achieve the desired fixation of a portion of a spine.

In a typical procedure where a bone hook is used, the bone hook is first loosely secured to a spinal fixation rod. The blade portion of the hook is then hooked around a desired portion of the vertebra, and once properly positioned, the bone hook is locked in place with respect to the spinal fixation system by tightening a nut or similar type of locking mechanism to securely interconnect the hook and the fixation rod. Often, it can be difficult to insert the hook under a vertebra because of the limited space that is available for a surgeon to work. Moreover, it can be difficult to maintain the position of the hook relative to the vertebra before the hook is attached to the rod and particularly when the fixation rod is being inserted into the rod-receiving element of the hook assembly. This is because such hooks can be susceptible to slipping out of place during placement and/or installation.

For procedures that require a vertebral body to be shifted or moved into a corrected position for engagement with the hook, correctly positioning the hook can be difficult. For example, even a slight rotational or angular misalignment between the hook portion of a bone hook and a vertebra to be engaged with the hook portion can make it difficult to engage the hook portion with the vertebra and mount the bone hook to a fixation rod. This problem can be compounded where plural hooks are used to engage sequential vertebra along a misaligned spinal curvature. In particular, it can be difficult to simultaneously position each hook in a position for connection to the spinal rod. Overcoming this difficulty may require the time consuming and difficult tasks of reshaping the rods or repositioning the hooks, each of which can require considerably longer operating time.

### SUMMARY

The present invention, according to one embodiment, is a bone hook for anchoring a spinal fixation rod to a vertebra. The bone hook includes a hook portion having a bone receiving region capable of receiving and engaging a portion of a bone. The bone hook further includes an auxiliary bone engaging element with a bone engaging tip. The auxiliary bone engaging element can be moved with respect to the hook portion, such that the bone engaging tip can be extended into the bone receiving region of the hook portion to contact a bone positioned in the bone receiving region.

The present invention, according to another embodiment, is a method for engaging a vertebra with a bone hook. In general, the method includes the steps of positioning a vertebra within a bone receiving region of a bone hook, extending a bone engaging tip of an auxiliary bone engaging element into the vertebra receiving region, and contacting the vertebra with the bone engaging tip. The bone hook includes a head portion that can be attached to a spinal rod of a spinal fixation system.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### BRIEF DESCRIPTION OF THE DRAWINGS

The accompanying drawings, which are incorporated in and constitute a part of this application, illustrate several aspects of the invention and together with a description of the embodiments serve to explain the principles of the invention. A brief description of the drawings is as follows:

FIG. 1 is a schematic side view of one embodiment of a spinal fixation system of the invention showing a bone hook engaged with a vertebra;

FIG. 2 is a perspective view of the bone hook of FIG. 1;

FIG. 3 is a partial cross-sectional view of the spinal fixation system of FIG. 1;

FIG. 4 is an exploded view in partial cross-section of the bone hook of FIG. 3;

FIG. 5 is a perspective view of an exemplary bone hook according to another embodiment of the present invention;

FIG. 6 is a side view of another bone hook assembly of the invention;

FIG. 7 is a top view of the body of the bone hook assembly of FIG. 6;

FIG. 8 is a side view of the body of the bone hook assembly of FIG. 6;

FIG. 9 is a bottom view of the body of the bone hook assembly of FIG. 6;

FIG. 10 is a side view of the hook of the bone hook assembly of FIG. 6;

FIG. 11 is a side view of another bone hook assembly of the invention;

FIG. 12 is a top view of the body of the bone hook assembly of FIG. 11;

FIG. 13 is a side view of the body of the bone hook assembly of FIG. 11;

FIG. 14 is a side view of the hook of the bone hook assembly of FIG. 11;

FIG. 15 is a cross-sectional view of the body shown in FIG. 12 taken along the line 15-15;

FIG. 16 is a cross-sectional view of the body shown in FIG. 15 taken along the line 16-16

FIG. 17 is a side view of another bone hook assembly of the invention;

FIG. 18 is a top view of the body of the bone hook assembly of FIG. 17;

FIG. 19 is a side view of the body of the bone hook assembly of FIG. 17;

FIG. 20 is a cross-sectional view of the body shown in FIG. 18 taken along the line 20-20;

FIG. 21 is a cross-sectional view of the body shown in FIG. 20 taken along the line 21-21; and

FIG. 22 is as side view of an alternative bone hook assembly according to the present invention.

### DETAILED DESCRIPTION

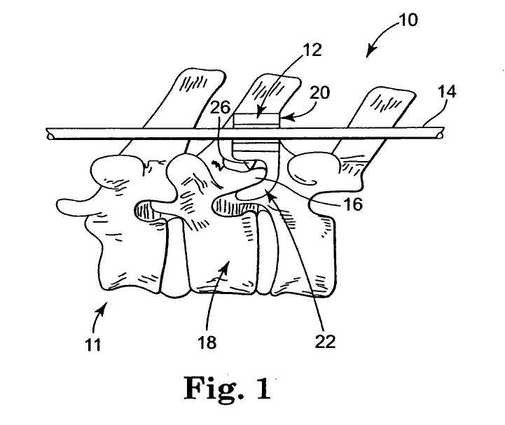

Referring to FIG. 1, a spinal fixation system 10 according to one embodiment of the present invention is shown with respect to a portion of a spine 11. As shown, the spinal fixation system 10 includes a bone hook 12 that is attached to a spinal fixation rod 14. Although not illustrated, the spinal fixation rod 14 can be secured to one or more additional vertebra by using any desired bone engaging components, such as those described in greater detail below, as well as by using as any known or developed hooks, bolts, wires, or screws, or the like, thereby providing a rigid supporting structure for the bone hook 12. As shown, the bone hook 12 is engaged with a lamina 16 of a vertebra 18 for holding or supporting the vertebra 18 in a desired position. For the purposes of the present discussion, bone hooks are discussed and illustrated with the understanding that hooks that engage with other portions of a vertebra and other spinal fixation systems may be used and can benefit from the present invention. For example, U.S. Pat. No. 6,050,997 granted to Mullane on Apr. 18, 2000 and assigned to the Assignee of the present invention describes typical spinal fixation systems (which disclosure is fully incorporated herein by reference).

Referring to FIGS. 1-4 collectively, one embodiment of the bone hook 12 of the present invention may include a head portion 20 and a hook portion 22. Also, as illustrated, the bone hook 12 may include a screw 24, which is described in greater detail below. Generally, however, in one embodiment of the present invention, the screw 24 is provided so that a bone engaging tip 26 of the screw 24 can extend into a bone receiving region 28 of the hook portion 22 and function to engage with the lamina 16. Moreover, in another embodiment of the present invention, when the head portion 20 and the hook portion 22 are formed as separate components, as in the illustrated embodiment, the screw 24 may also function to attach and lock the head portion 20 to the hook portion 22 as described in greater detail below. That is, the head portion 20 and the hook portion 22 can be rotated with respect to each other and locked in place with the screw 24 for adjusting the relative orientation of the head portion 20 and hook portion 22 during installation in a patient.

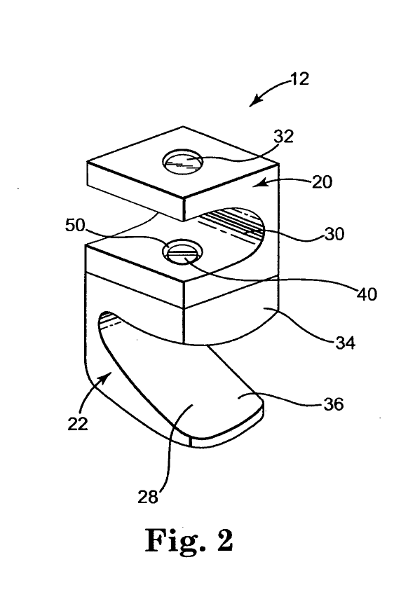

The head portion 20 of the bone hook 12, as shown, is designed to receive and engage the spinal fixation rod 14. For example, as can best be seen in FIG. 2, the head portion 20 may include a U-shaped slot 30 that can receive the spinal fixation rod 14 from a transverse or side direction with respect to a spine (left or right with respect to the human body). It is noted, however, that the head portion 20 can be configured to receive the spinal rod 14 from any direction such as from an anterior or posterior direction (front to back with respect to the body). That is, the head portion 20 does not need to use a slot and may use a bore, for example, or any other means to connect the head portion 20 to the spinal fixation rod 14. In any case, the head portion 20 is designed so that the head portion 20 includes the capability to attach the bone hook 12 to the spinal fixation rod 14.

In the illustrated embodiment, a set screw 32 may be provided, as shown, for securing the spinal fixation rod 14 within the slot 30 as described below. It is noted, however, that the head portion 20 may comprise any device for securing the head portion 20 to the spinal fixation rod 14. Such devices may include, for example, clamping or mechanical securing devices. Moreover, devices that attach the head portion 20 directly to the spinal fixation rod 14 as well as those that attach the head portion 20 indirectly such as by using transverse connectors and the like can be used.

The bone-receiving region 28 of the hook portion 22 may be designed so that the bone receiving region 28 is capable of receiving and engaging a desired portion of a vertebra such as the lamina 16. For example, the hook portion 22, as shown, has an upper portion 34 and a lower blade portion 36. The size and shape of the bone receiving region 28 is generally determined and selected according to the dimensions of the target site of implantation, such as, in the illustrated example, the lamina 16. Thus, the bone receiving region 28 may have substantially a C-shape as illustrated. It is also noted that the upper and lower portions 34 and 36 respectively, can be integrally formed (for example, as a monolithic structure as illustrated), or alternatively, they can comprise plural elements that have been mated together using a suitable technique such as mechanical fastening or the like.

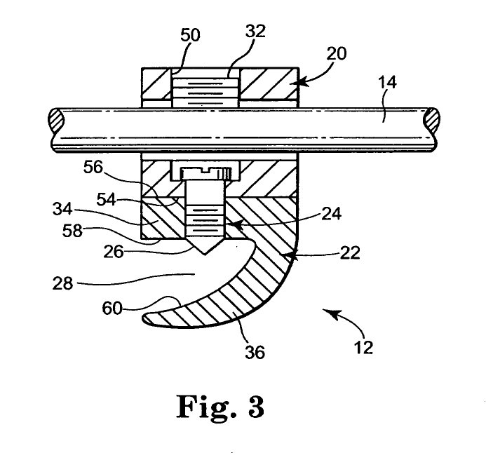

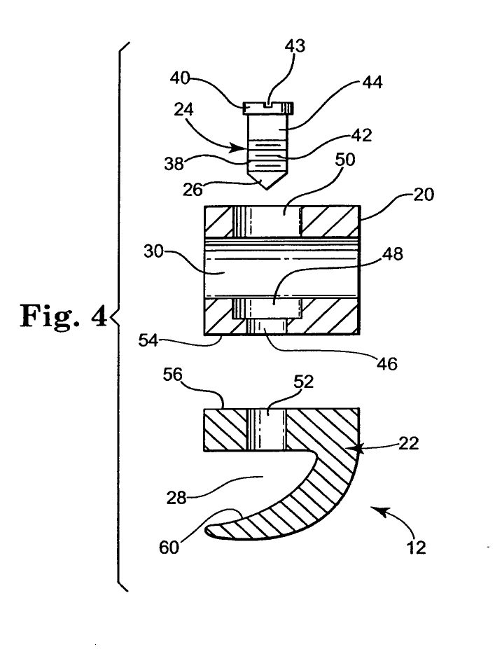

In one embodiment, illustrated in FIG. 3, the bone engaging tip 26 of the screw 24 may be configured to extend at least partially into the bone receiving region 28. For example, in one embodiment, the screw 24 may be tightened to clamp a surface 54 of the head portion 20 to a surface 56 of the hook portion 22 (see FIG. 4), and when the surfaces 54 and 56 are so clamped together, the bone engaging tip 26 extends beyond an inner surface 58 of the hook portion 22 by a predetermined amount, which can be selected as described below. In such an embodiment, the screw 24 may function as an auxiliary bone engaging element to both lock the hook portion 22 to the head portion 20 and to provide a bone engaging function with the bone engaging tip 26. That is, the hook portion 22 itself provides a primary supporting or engaging function and the bone engaging tip 26 of the screw 24 provides an auxiliary or assisting engaging function. In another embodiment (not shown) the bone engaging function and the locking function (locking of the head portion 20 and the portion 22) of the screw 24 are provided by separate and distinct devices.

In one embodiment, the screw 24 may be sized such that the bone engaging tip 26 can extend into the bone receiving region 28 a distance sufficient to positively engage a vertebra positioned therein. The bone engaging tip 26 can be driven into contact with a vertebra to provide such engagement. Accordingly, the bone engaging tip 26 may provide a clamping action between the bone engaging tip 26 and an inner surface 60 of the hook portion 22 when a vertebra is positioned therein. Such engagement may help to hold the hook portion 22 with respect to a vertebra positioned therein by the clamping action between the bone engaging tip 26 and the surface 60, which provides a clamping force. Such clamping force may be determined empirically, for example, and/or by considering factors including the characteristics of the particular bone (hardness, softness, and/or shape, for example) at the implant site, the amount of holding force desired, and the like. The distance that the bone engaging tip 26 extends into the bone receiving region 28 may thus be selected by considering factors such as the desired clamping force, geometry of a target vertebra, and the softness or hardness of the bone. Regarding the surface 60, it is contemplated that the surface 60, or any portion thereof, may include friction enhancing or gripping features such as roughened, scored, knurled, or serrated regions or portions.

In the embodiment shown in FIGS. 1-4, the bone engaging tip 26 comprises a pointed tip. In other embodiments, the bone engaging tip 26 may comprise any desired structure or shape that can positively engage with a vertebra in accordance with the present invention. Such engagement may comprise pressing the bone engaging tip 26 against the surface of the vertebra in order to provide a desired holding force. For example, the bone engaging tip 26 may comprise a flat surface for engaging with the vertebrae, which surface may be roughened, scored, knurled, or otherwise modified to improve engagement or holding between the bone engaging tip 26 and a vertebra. The bone engaging tip 26 may also comprise rounded, bullet nosed, pyramidal, wedge, knife edge, or serrated shapes and/or structures. Any structure that can provide at least some engagement or frictional resistance between the bone engaging tip 26 and the surface of a bone can be used.

Referring to FIG. 4 in particular, an exploded view of the bone hook 12 is shown. As shown in FIG. 4, the screw 24 may include a shaft 38 and a head 40. The shaft 38, as shown, may include a threaded portion 42 and an unthreaded portion 44. In other embodiments, the unthreaded portion 44 may be omitted. As illustrated, the head 40 may include a slot 43 that can be used to drive the screw 24 in accordance with the invention as described below. For example, a screwdriver can be used to engage with the slot 42 to drive the screw 24. It is noted, however, that the head 40 may include any desired feature for providing such driving function such as a socket or other engaging means.

As illustrated, the head portion 20 of the bone hook 12 includes an unthreaded bore 46 and a concentric countersunk bore 48 which are used to attach the head portion 20 to the hook portion 22 with the screw 24. The bores 46 and 48 are configured to allow the screw 24 to be recessed into the head portion 20 so that it does not interfere with the spinal rod 14 when positioned in the slot 30. The unthreaded bore 46 has a diameter that is slightly larger than the diameter of the shaft 38 so that the shaft 38 can freely pass through the unthreaded bore 46 and so that the shaft 38 and head portion 20 can freely rotate with respect to each other when the shaft 38 is positioned within the unthreaded bore 46. The countersunk bore 48 also has a diameter and depth that are large enough to at least partially receive the head 40.

As shown in FIG. 4, in one embodiment, the head portion 20 of the bone hook 12 may also include a hole 50 adapted to receive the set screw 32 (see FIGS. 2 and 3) so that the set screw 32 can be used to secure the bone hook 12 to the spinal fixation rod 14. As illustrated in FIGS. 2 and 3, the set screw 32 can be tightened within the hole 50 so that the set screw 32 clamps the spinal fixation rod 14 within the slot 30. It is noted, however, that the head portion 20 can be secured to the spinal fixation rod 14 in any manner. For example, a plurality of set screws can be used. Additionally, a bore can be used instead of a slot. In other embodiments, other mechanical devices that perform a clamping or fixing function can be used.

The hole 50 may be designed to be capable of allowing the head 40 of the screw 24 to pass there through. As such, the screw 24 can be used to attach the head portion 20 to the hook portion 22 as is shown in FIG. 3. This type of approach could be useful if a bore is used instead of the slot 30, for example. As shown in FIG. 3, in one embodiment, when the head 40 is seated in the bore 48, the unthreaded portion 44, if present, is positioned substantially within the bore 46. In another embodiment, the shaft 38 may be threaded along substantially its entire length and the unthreaded bore 46 is sized so as to not interfere with the shaft 38 and to allow the shaft 38 to translate and rotate with respect to the unthreaded bore 46.

Alternatively, the head portion 20 may be designed so that the screw 24 does not need to pass through the tapped hole 50 in order to attach the head portion 20 to the hook portion 22. For example, the screw 24 can be designed so that the screw 24 can be loaded into the bore 46 by placing the screw 24 within the slot 30. In other words, the screw 24 can be designed so that it can be inserted into the bore 46 by passing through a portion of the slot 30.

As further shown in FIG. 4, the hook portion 22 may include a threaded opening 52 capable of receiving the threaded portion 42 of the screw 24. In such an embodiment, the threads of the threaded portion 42 are configured so that the threaded portion 42 can be screwed into the threaded opening 52. The screw 24 can thus be used to attach the head portion 20 to the hook portion 22 and to rotationally lock the hook portion 22 with respect to the head portion 22.

Also, the surface 54 and/or the surface 56 may optionally include friction enhancing features (not shown). Such friction enhancing features can be used to help to improve locking engagement (rotationally or slidingly) between the head portion 20 and the hook portion 22. For example, one or both of surfaces 54 and 56 may be roughened, scored, or provided with serrations or the like that can increase frictional or gripping engagement between surfaces 54 and 56.

Referring back to FIG. 3, a cross-sectional view of the assembled bone hook 12 is shown. The bone hook 12 can be assembled by positioning the hook portion 22 with respect to the head portion 20 so that a surface 54 of the head portion 20 contacts a surface 56 of the hook portion 22. The head portion 20 may also be positioned so that the unthreaded bore 46 of the head portion 20 is axially aligned with the threaded opening 52 of the hook portion 22. The screw 24 may be passed through the tapped hole 50 and used to attach the head portion 20 to the hook portion 22. The threaded portion 42 of the shaft 38 may be threaded or screwed into the threaded opening 52 and driven by the slot 43 (such as with a screwdriver or the like) to seat the head 40 within the bore 48. Additionally, the head portion 20 may rotate with respect to the hook portion 22 until the screw 24 is further tightened to lock the hook portion 22 to the head portion 20. In another embodiment, a separate device or mechanism may be used to attach the hook portion 22 to the head portion 20. For example a screw or bolt or other mechanical fastening device, distinct from the screw 24, that allows the hook portion 22 to rotate with respect to the head portion 20 and that can lock the rotation of the hook portion 22 with respect to the head portion 20, may be used.

It is noted that the unthreaded bore 46 and the bore 48 could be functionally replaced by slots or other structures that extend through an edge or side of the head portion 20. In particular, it is contemplated that the bone hooks of the present invention may be designed so that the screw 24 can be accessed when the spinal rod 14 is positioned in the slot 30. That is, the position of the screw 24 may be offset from the position of the spinal rod 14 in order to provide clearance between the screw 24 and the spinal rod 14.

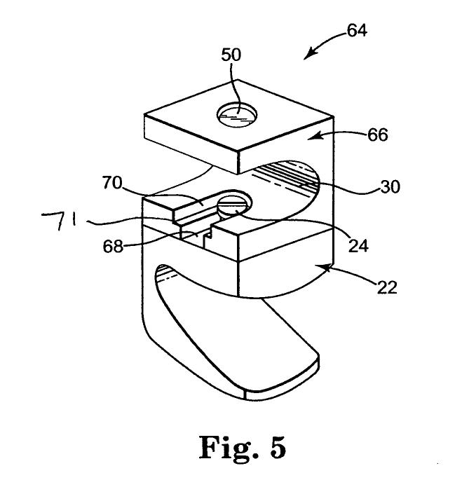

Referring to FIG. 5, a bone hook 64 according to another embodiment of the present invention is illustrated. As shown in FIG. 5, the bone hook 64 includes the hook portion 22 and screw 24, as described above, and also includes a head portion 66 similar to the head portion 20 described above. The head portion 66 differs from the head portion 20 in that the head portion 66 includes slots 68 and 70 and a shoulder 71. As such, the screw 24 may be at least partially threaded into the threaded opening 52 (See FIG. 4) of the hook portion 22 and the slots 68 and 70 may be mated with the screw 24. The hook portion 22 may then be rotated with respect to the head portion 66 (or additionally translated along the slot) in order to position the hook portion 22 as desired. The screw 24 can then be tightened against the shoulder 71 in order to clamp the hook portion 22 in place with respect to the head portion 66. In one embodiment, the shoulder 71 may include a recessed portion (not shown) sized and shaped to partially receive the screw head 40 to more positively retain the screw 24 in the slots 68 and 70.

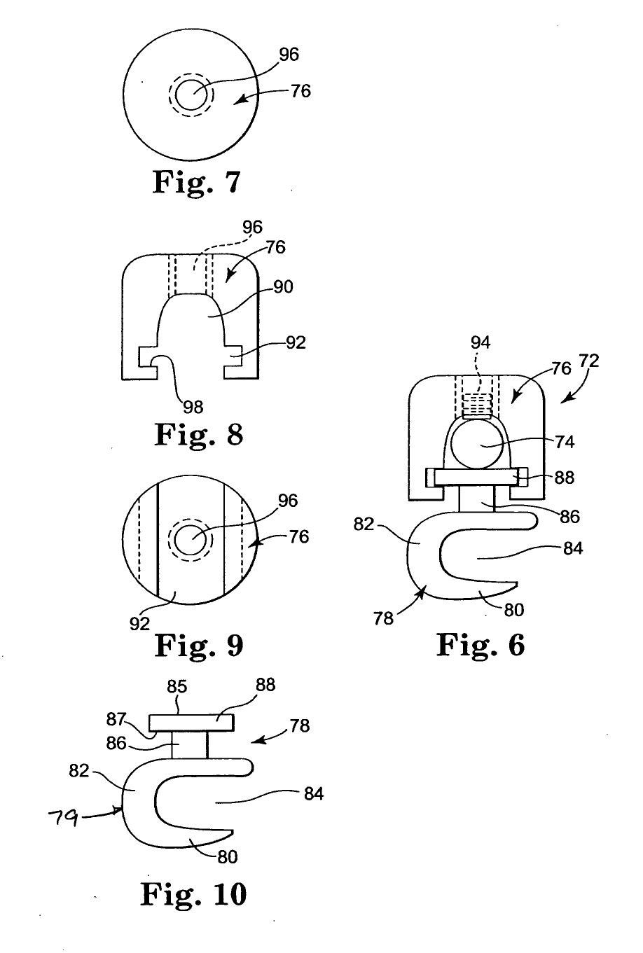

FIG. 6 shows a bone hook assembly 72 according to another embodiment of the present invention. In FIG. 6, the bone hook assembly 72 of the invention is shown attached to a spinal rod 74. As shown, the hook assembly 72 may include a body 76 and a hook 78. The body 76 is shown in further detail in FIGS. 7-9 (as removed from the spinal rod 74). FIGS. 7 and 9 show top and bottom views of the body 76, respectively, while FIG. 8 shows a side view of the body 76. Also, in FIG. 10 the hook 78 is illustrated.

Referring to FIG. 10, the hook 78 may include a bone-engaging portion 79, a shaft 86, and a head portion 88. As illustrated, the shaft portion 86 extends from the bone engaging portion 79 and is connected to the head portion 88. The bone engaging portion 79 includes a blade portion 80 and a shank portion 82 that define a bone receiving region 84. As described above, the bone receiving region 84 can be provided to engage with any desired portion of a vertebral bone such as a laminar portion of a vertebra.

Referring to FIGS. 7-9 collectively, the body 76 may include an opening 90 adapted to receive the spinal rod 74. In the illustrated embodiment, the body 76 may also include an opening 92 for receiving the head portion 88 of the hook 78. As shown, the opening 92 is provided as a slot or channel that passes through the body 76. The opening 92 may function as a seat for the head portion 88 of the hook 78 as is described in further detail below. Thus, it is noted that the opening 92 does not need to pass all the way through the body 76 as illustrated. The opening 92 may be provided through any portion of the body 76 as desired. For example, the opening 92 may be provided through a portion of the body 76 in order to define a stop or to define a desired location for the hook 78 with respect to the body 76.

As shown, the opening 92 is provided so that the head portion 88 of the hook 78 may be inserted into the opening 92 in a direction that is generally parallel to a direction of extension of the spinal rod 74. It is noted that the body 76 may be designed so that the head portion 88 of the hook 78 may be inserted into the opening 92 from any desired direction or angle with respect to the spinal rod 74. That is, the opening 92 may be formed at any desired direction or angle in the body 76. For example, the body 76 may be designed to receive the head portion 88 of the hook 78 in a direction that is generally perpendicular to the spinal rod 74. As such, the body 76 may be side-loading or transverse loading.

Referring to FIG. 6, the hook 78 is shown assembled with the body 76. As shown, the head portion 88 of the hook 78 may be positioned in the opening 92 of the body 76. The head portion 88 is rotatable with respect to the body 76 such that the hook 78 may be positioned as desired with respect to the body 76. As such, the head portion 88 may be provided as a disk that can rotate within the opening 92, as shown.

In use, the body 76 and hook 78 may be attached to the spinal fixation rod 74 in any desired order. For example, the spinal fixation rod 74 may be positioned in the opening 90 of the body 76. Next, the head portion 88 of the hook 78 may be slid into the opening 92 or otherwise loaded into the opening 92. The hook 78 may be rotated to a desired position such that the bone receiving region 84 of the hook 78 engages a vertebra. A set screw 94 may be provided in a tapped hole 96 of the body 76, and the set screw 94 may be driven into contact with the spinal fixation rod 74 thereby driving the spinal fixation rod 74 into contact with a surface 85 of the head portion 88 of the hook 78. By driving the spinal fixation rod 74 into contact with a surface 85 of the head portion 88, a surface 87 of the head portion 88 is driven into contact with a surface 98 of the opening 92 of the body 76. As such, the head portion 88 may be clamped or fixed with respect to the body 76. That is, the surface 87 of the head portion 88 may be forced against the surface 98 of the opening 92 to provide sufficient frictional resistance between the surface to hold the head portion 88 in a fixed position with reference to the body 76. If desired, any of the surfaces 85, 87, and 98 may include friction enhancing or gripping features such as roughened, scored, knurled, or serrated regions or portions. Additionally, the surface 98 may optionally include a recessed portion (not shown) sized and shaped to partially receive the head portion 88 and thereby further prevent the head portion 88 from sliding out of the opening 92.

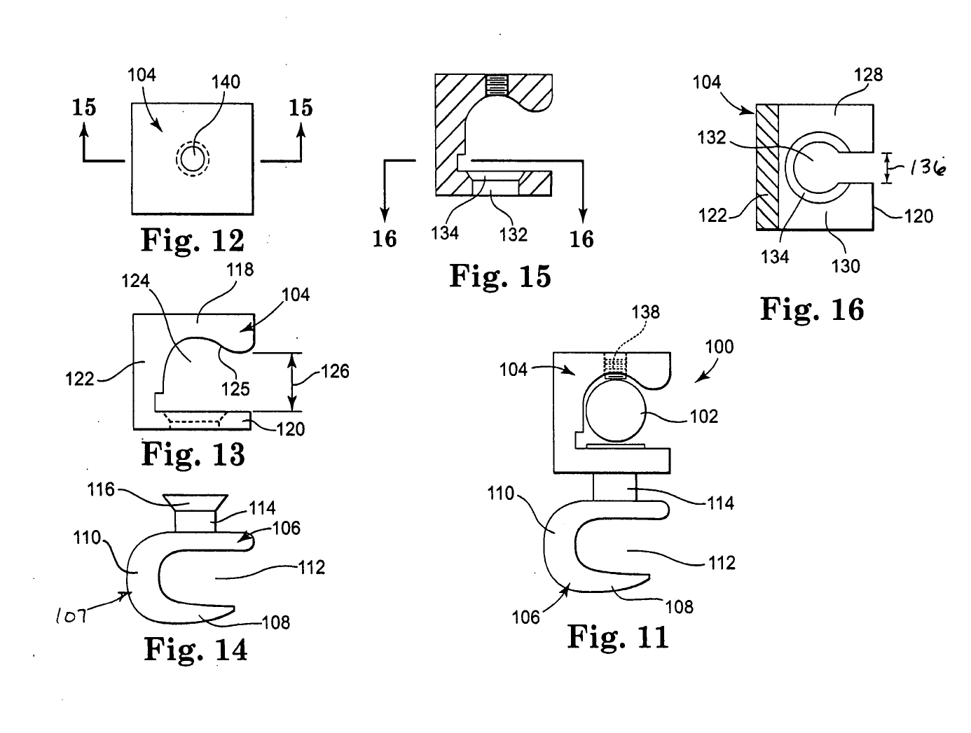

With reference to FIG. 11, another hook assembly 100 of the invention is shown attached to a spinal rod 102. As shown, the hook assembly 100 includes a body 104 and a hook 106. The body 104 is shown in further detail in FIGS. 12, 13, 15, and 16. FIGS. 12 and 13 show top and side views of the body 104, respectively. Also, FIGS. 15 and 16 show cross-sectional views of the body 104 and in FIG. 14 the hook 106 is illustrated.

Referring to FIG. 14, the hook 106 includes a bone engaging portion 107, a shaft portion 114, and a head portion 116. The shaft portion 114 extends from the bone engaging portion 107 and is connected to the head portion 116. The bone engaging portion 107 includes a blade portion 108 and a shank portion 110 that define a bone receiving region 112. As described above, the bone receiving region 112 can be designed to engage with any desired portion of a vertebral body such as a laminar portion.

As shown in FIG. 13, the body 104 may include an upper portion 118 and a lower portion 120 that are spaced apart by a side portion 122 (upper, lower, and side are indicated for reference within the Figure only). Accordingly, the body 104 may have a C-shape when viewed from a side as shown in FIG. 13. The upper portion 118 and lower portion 120 are spaced apart to define a region 124 for receiving the spinal fixation rod 102 as illustrated in FIG. 11. As shown, the region 124 is partially defined by a surface 125, which, in the illustrated embodiment, is designed to follow a curve. For example, the body 104 can be designed to receive the spinal fixation rod 102 by a snap fit as described in more detail below. Accordingly, the body 104 may be designed to be elastically deformable as explained below. However, it is understood that the surface 125 does not need to be curved and can be a flat surface.

During surgical implantation, the spinal fixation rod 102 may be positioned in the region 124 by inserting the spinal fixation rod 102 through a gap 126 between respective ends of the upper and lower portions 118 and 120, respectively. This allows the body 104 to be implanted after the spinal rod 102 is fixed in position, if desired. The body 104 may be designed to provide a snap fit between the upper and lower portions 118 and 120 and the spinal fixation rod 102. For example, the gap 126 may be less than the diameter of the spinal fixation rod 102 so that the upper and lower portions 118 and 120 elastically deflect away from each other when the spinal fixation rod 102 is pushed through the gap 126. Thus, as mentioned above, the surface 125 may be formed as a curved surface. In one embodiment, the surface 125 has a radius of curvature that is substantially the same as the radius of the spinal fixation rod 102. Alternatively, the surface 125 may have a radius of curvature that is slightly larger or slightly smaller than the radius of the spinal fixation rod 102. As such, the spinal fixation rod 102 can be captured in the region 124 when pushed through the gap 126. However, it is contemplated that the body 104 may be designed so that the gap 126 is large enough to allow the spinal fixation rod 102 to pass through to the region 124 without substantial interference with the upper and lower portions 118 and 120. As such, the gap 126 may be slightly larger than the diameter of the spinal fixation rod 102. In other words, a snap-fit design for the body 104 does not need to be used.

With reference to FIG. 16, the lower portion 120 of the body 104 may include spaced apart arms 128 and 130, an opening 132, and a seat 134. The opening 132 and the seat 134 are shown in more detail in FIG. 15. As shown, the opening 132 may be sized and shaped to receive the shaft portion 114 of the hook 106 such that the shaft portion 114 is rotatable within the opening 132. The seat 134 may be designed to cooperate with the head portion 116 of the hook 106 so that the head portion 116 can rotate in the seat 134. Thus, as shown, the seat 134 and the head portion 116 are tapered such that the head portion 116 can mate within the seat 134. In the embodiment illustrated in FIG. 16, the taper of the head portion 116 and seat 134 allow the head portion 116 to rotate in the seat 134. In other embodiments, the head portion 116 and the seat 134 may be designed so that the head portion 116 can angularly move with respect to body 104. For example, the head portion 116 and seat 134 may be formed to have a cylindrical shape so that the head portion 116 could angularly move with respect to the body 104. Alternatively, the head portion 116 and seat 134 may be formed to have a spherical shape so that the head portion 116 may angularly and rotationally move with respect to the body 104.

Further referring to FIG. 16, the arms 128 and 130 are spaced apart by a gap 136. As shown, the gap 136 allows a side loading of the hook 106 (perpendicular to the direction of extension of the spinal rod 102). This allows the hook 106 to be assembled to the body 104 either before or after the body 104 is positioned with respect to the spinal rod 102. The arms 128 and 130 may be designed so that the gap 136 provides a snap fit between the shaft portion 114 of the hook 106 and the arms 128 and 130. In such embodiments, the gap 136 is less than the diameter of the shaft portion 114, and when the shaft portion 114 is pushed through the gap 136, the arms 128 and 130 elastically move apart from each other to allow the shaft portion 114 to pass through the gap 136 to the opening 132.

In use, the body 104 and hook 106 may be attached to the spinal fixation rod 102 in any desired order. For example, the spinal fixation rod 102 may be positioned in the region 124 of the body 104 by pushing the spinal fixation rod 102 through the gap 126 (if a snap fit design is used). The spinal fixation rod 102 may also be positioned in the region 124 by inserting an end of the spinal fixation rod 102 into the region 124. The shaft portion 114 of the hook 106 may be positioned into the opening 132 by pushing the shaft portion 114 though the gap 136. The hook 106 may be rotated to a desired position such that the bone receiving region 112 of the hook 106 engages a vertebra. A set screw 138 may be provided in a tapped hole 140 of the body 104, and the set screw 138 may be driven into contact with the spinal fixation rod 102 thereby driving the spinal fixation rod 102 into contact with the head portion 116 of the hook 106. By driving the spinal fixation rod 102 into contact with the head portion 116, the head portion 116 is driven into contact with the seat 134 of the body 104. As such, the head portion 116 may be clamped or fixed with respect to the body 104 in a similar manner as described above with respect to hook assembly 72 shown in FIG. 6.

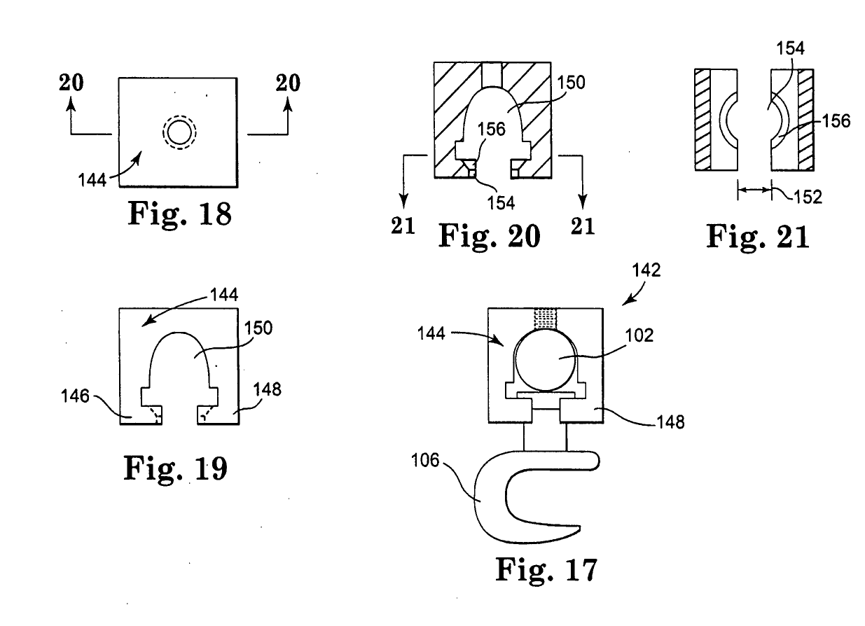

With reference to FIG. 17, another hook assembly 142 of the present invention is shown attached to the spinal rod 102. As shown, the hook assembly 142 includes a body 144 and the hook 106 described above with respect to the bone hook assembly 100. The body 144 is shown in further detail in FIGS. 18-21. FIGS. 18 and 19 show top and side views of the body 144, respectively, and FIGS. 20 and 21 show cross-sectional views of the body 144.

As can be seen in FIG. 19, the body 144 may include spaced apart legs 146 and 148 that define a region 150 for receiving the spinal rod 102. The legs 146 and 148 are spaced apart by a gap 152 as shown. The body 144 may also include an opening 154 for receiving the shaft portion 114 of the hook 106 and a seat 156 for receiving the head portion 116 of the hook 106 as described above. As such, the hook 106 may snap fit between the legs 146 and 148 in a similar manner to that described above with respect to the bone hook assembly 100. That is, the body 144 is designed so that the legs 146 and 148 can elastically spread apart to receive the head portion 116 and retain the head portion 116 within the seat 156.

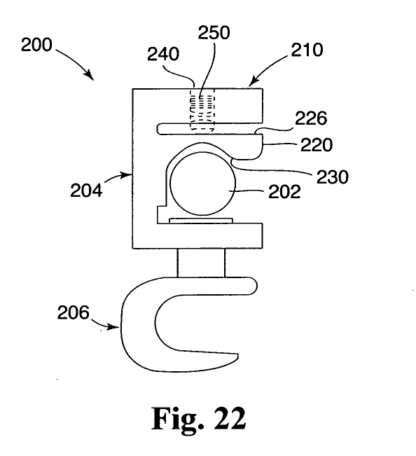

FIG. 22 depicts an alternative bone hook assembly 200 according to the present invention attached to a spinal rod 202. The bone hook assembly 200 includes a head 204 and a hook 206, and is in other respects similar to the assembly 100 described above, except that the head 204 includes an upper member 210 and a clamping member 220 having an upper surface 226 and a lower surface 230. The upper member 210 includes a threaded hole 240. A set screw 250 adapted to threadingly engage the threads of the hole 240 may be driven into contact with the upper surface 226 thereby deflecting the clamping member 220 downward. The set screw 250 is sized such that the lower member 220 can be deflected an amount sufficient to drive the lower surface 230 into contact with the spinal rod 202, thereby clamping the spinal rod 202 to the bone screw assembly 200.

The bone hooks of the present invention may be constructed from any desired material that is biologically compatible and suitable for medical applications. One exemplary material from which the bone hooks may be made is stainless steel. Other materials from which the bone hooks can be constructed include titanium as well as medical grade alloys and the like.

The present invention has now been described with reference to certain specific embodiments. The foregoing detailed description has been given for clarity of understanding. Others may recognize that changes can be made in the described embodiments without departing from the scope and spirit of the invention. Thus, the scope of the present invention should not be limited to the exact details and structures described herein.

What is claimed is:

1. A bone hook for anchoring a spinal fixation rod to a vertebra, the bone hook comprising:
a hook portion having a bone receiving region that can receive and engage at least a portion of a bone; and
an auxiliary bone engaging element comprising a bone engaging tip, the auxiliary bone engaging element moveable with respect to the hook portion such that the bone engaging tip can be extended at least partially into the bone receiving region of the hook portion to contact a bone positioned in the bone receiving region of the hook portion.

2. The bone hook of claim 1 further comprising a head portion coupled to the hook portion and having a rod receiving region.

3. The bone hook of claim 2 wherein the rod receiving region comprises a slot provided in the head portion.

4. The bone hook of claim 2 further comprising a securing device adapted for securing the head portion to a rod when a rod is positioned in the rod receiving region.

5. The bone hook of claim 4 wherein the securing device comprises a threaded fastener that can pass through a wall of the head portion for securing the head portion to a rod.

6. The bone hook of claim 2 wherein the hook portion is movable with respect to the head portion.

7. The bone hook of claim 6 wherein the hook portion is rotatable with respect to the head portion.

8. The bone hook of claim 2 wherein the head portion is separably attached to the hook portion with the auxiliary bone engaging element.

9. The bone hook of claim 8 wherein the auxiliary bone engaging element comprises a fastener having a threaded portion and a head portion.

10. The bone hook of claim 9 wherein the threaded portion of the fastener engages with a threaded portion of the hook portion and the head portion of the fastener engages with the head portion of the bone hook for separably attaching the head portion of the bone hook to the hook portion.

11. The bone hook of claim 1 wherein the auxiliary bone engaging element further comprises a threaded portion that can engage with a threaded portion of the hook portion for driving the bone engaging tip into the bone receiving region of the hook portion.

12. The bone hook of claim 1 wherein the bone engaging tip comprises a flat surface for engaging with a bone.

13. The bone hook of claim 1 wherein the bone engaging tip comprises a conically shaped surface for engaging with a bone.

14. The bone hook of claim 1 wherein the bone engaging tip comprises a spherically shaped surface for engaging with a bone.

15. The bone hook of claim 1 wherein the bone receiving region has a size and a shape adapted to engage a lamina of a vertebra.

16. A system for anchoring a spinal fixation rod to a vertebra, the system comprising:
a bone hook having a bone engaging portion and a head portion; and
a body having a rod receiving region and a seat adapted to slidingly receive the head portion of the bone hook so that the head portion of the bone hook can rotate in the seat when positioned therein.

17. The system of claim 16 wherein the seat of the body comprises a slot provided through at least a portion of the body.

18. The system of claim 17 wherein the head portion of the bone hook comprises a disk capable of being slidingly received by the seat of the body.

19. A system for anchoring a spinal fixation rod to a vertebra, the system comprising:
a bone hook having a bone engaging portion and a head portion; and
a body having a rod receiving region and first and second members extending from the body and flanking a seat that can receive the head portion of the bone hook such that the head portion of the bone hook can rotate in the seat when positioned therein, the first and second members having ends elastically deflectable away from each other to snap fit around the head portion of the bone hook.

20. The system of claim 19 wherein the body further comprises a securing device adapted for securing the body to a rod when a rod is positioned in the rod receiving region.
